SEQUENCE LISTING (15 sequences)

SEQ ID NO: 1 - DNA, 129 bp, Artificial Sequence
  Chemically synthesized Aptazyme
taatcttaccccggaattatatccagctgcatgtcaccatgcagagcagactatatctccaacttgttaaagcaagttgtctatcgtttcgagtcacttgaccctactccccaaagggatagtcgttag

SEQ ID NO: 2 - DNA, 131 bp, Artificial Sequence
  Chemically Synthesized Aptazyme
gcctgagtataaggtgacttatacttgtaatctatctaaacggggaacctctctagtagacaatcccgtgctaaattataccagcatcgtcttgatgcccttggcagataaatgcctaacgactatccctt

SEQ ID NO: 3 - DNA, 75 bp, Artificial Sequence
  Chemically Synthesized Aptazyme
gataatacgactcactatagggatcaacgctcagtagatgttttcttgggttaattgaggcctgagtataaggtg

SEQ ID NO: 4 - DNA, 89 bp, Artificial Sequence
  Chemically Synthesized Aptazyme
cttagctacaatatgaactaacgtagcatatgacgcaatattaaacggtagcattatgttcagataaggtcgttaatcttaccccggaa

SEQ ID NO: 5 - DNA, 131 bp, Artificial Sequence
  Chemically Synthesized Aptazyme
  misc_feature (77)..(77): n is a, c, t or g
  misc_feature (108)..(108): n is a, c, t or g
gcctgagtataaggtgacttatactagtaatctatctaaacggggaacctctctagtagacaatcccgtgctaaatnataccagcatcgtcttgatgcccttggcagntaaatgcctaacgactatccctt

SEQ ID NO: 6 - DNA, 101 bp, Artificial Sequence
  Chemically Synthesized Aptazyme
cttagctacaatatgaactaacgtagcatatgacgcaatattaaacggtagtattatgttcagataaggtcgttaatcttaccccggaattctatccagct

SEQ ID NO: 7 - RNA, 270 nt, Bacteriophage T4 (wild type)
  Group 1 theophylline-dependent (td) intron
uuggguuaauugaggccugaguauaaggugacuuauacuuguaaucuaucuaaacggggaaccucucuaguagacaaucccgugcuaaauuguaggacuggddcbacauaaaugccuaacgacuaucccuuuggggaguagggucaagugacucgaaacgauagacaacuugcuuuaagaaguuggagauauagucugcucugcauggugacaugcagcuggauauaauuccgggguaagauuaacgaccuuaucugaacauaaugcuac

SEQ ID NO: 8 - RNA, 82 nt, Artificial Sequence
  chemically synthesized GpITh1P6.131 aptamer construct
uaaacggggaaccucucuaguagacaaucccgugcuaaauuauaccagcaucgucuugaugcccuuggcagauaaaugccua

SEQ ID NO: 9 - RNA, 84 nt, Artificial Sequence
  chemically synthesized GpITh1P6.133 aptamer construct
uaaacggggaaccucucuaguagacaaucccgugcuaaauugauaccagcaucgucuugaugcccuuggcagcauaaaugccua

SEQ ID NO: 10 - RNA, 40 nt, Artificial Sequence
  chemically synthesized portion of P6 region of the Group I ribozyme (Part I)
uaaacggggaaccucucuaguagacaaucccgugcuaaau

SEQ ID NO: 11 - RNA, 30 nt, Artificial Sequence
  chemically synthesized Anti-theophylline aptamer
auaccagcaucgucuucaugcccuuggcag

SEQ ID NO: 12 - RNA, 10 nt, Artificial Sequence
  chemically synthesized portion of P6 region of the Group I ribozyme (Part II)
uaaaugccua

SEQ ID NO: 13 - DNA, 18 bp, Artificial Sequence
  chemically synthesized primer sequence
gcgactggacatcacgag

SEQ ID NO: 14 - RNA, 130 nt, Artificial Sequence
  chemically synthesized LI ligase aptazyme construct
ggacuucgguccagugcucgugcacuaggccguucgaccauguggguccgcugccagcggcaaucuggcaugcuaugcggaaccuucacaucuuagacaggagguuaggugccucgugauguccagucgc

SEQ ID NO: 15 - RNA, 96 nt, Artificial Sequence
  chemically synthesized modified LI ligase Aptazyme construct
  misc_feature (17)..(66): n is a, c, t or g
ggaccucggcgaaagcnnnnnnnnnnnnnnnnnnnnnnnnnnnnnnnnnnnnnnnnnnnnnnnnnngagguuaggugccucgugauguccagucgc

(19) United States
(12) Patent Application Publication
(10) Pub. No.: US 2007/0077571 A1
(43) Pub. Date: Apr. 5, 2007

(54) Methods and apparatus for identifying allosterically regulated ribozymes

(76) Inventors: Andrew D. Ellington, Austin, TX (US); Michael P. Robertson, Austin, TX (US); J. Colin Cox, Austin, TX (US); Timothy E. Riedel, Austin, TX (US); Eric A. Davidson, Austin, TX (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/436,720

(22) Filed: May 17, 2006

Related U.S. Application Data
(63) Continuation of application No. 09/666,870, filed on Sep. 20, 2000, now abandoned.
(60) Provisional application No. 60/212,097, filed on Jun. 15, 2000.

(51) Int. Cl. C12Q 1/68

(52) U.S. Cl. 435/6

(57) ABSTRACT

Compositions and methods are provided to use regulatable aptazymes for controlling gene expression and in assays to detect the presence of ligands or to detect activation by an effector of an aptazyme bound to a solid support such as a substrate or multi-well plate. Regulatable aptazymes are ribozymes that are allosterically regulated by an effector molecule. Also disclosed are compositions and methods for automating the selection procedures of the present invention.

This patent claims priority from provisional patent application Ser. No.: 60/212,097, entitled “Aptazymes for Genetic Regulatory Circuits”, by Andrew D. Ellington, et al., filed Jun. 15, 2000.

### BACKGROUND OF THE INVENTION

The United States Government may own certain rights in this invention under DARPA Grant No.: N65236-98-1-5413 and MURI Grant No.: DAAD19-99-1-0207.

### FIELD OF THE INVENTION

The present invention relates generally to the field of ribozymes and in particular to aptazymes or allosteric, regulatable ribozymes that modulate their kinetic parameters in response to the presence of an effector molecule.

Ribozymes or RNA enzymes are oligonucleotides of RNA that can act like enzymes by catalyzing the cleavage of RNA molecules. Generally, ribozymes have the ability to behave like an endoribonucleases. The location of the cleavage site is highly sequence specific, approaching the sequence specificity of DNA restriction endonucleases. By varying conditions, ribozymes can also act as polymerases or dephosphorylases.

Ribozymes were first described in connection with Tetrahymena thermophilia. The Tetrahymena rRNA was shown to contain an intervening sequence (IVS) capable of excising itself out of a large ribosomal RNA precursor. The IVS is a catalytic RNA molecule that mediates self-splicing out of a precursor, whereupon it converts itself into a circular form. The Tetrahymena IVS is more commonly known now as the Group I Intron.

Regulatable ribozymes have been described, wherein the activity of the ribozyme is regulated by a ligand binding moiety. Upon binding the ligand, the ribozyme activity on a target RNA is changed. The ligand-binding portion is an RNA sequence capable of binding a ligand such as an organic or inorganic molecule, or even a prodrug. The regulatable ribozymes described to date target bind, e.g., a first target sequence and the enzymatic activity is brought to bear on a separate RNA molecule for cleavage.

Ribozymes provide a mechanism to control the expression of genes in vitro and in vivo and therefore the expression of genes in patients. As such, ribozyme-based gene therapies using ribozymes may provide great medical benefits.

### SUMMARY OF THE INVENTION

The present invention provides an allosterically regulatable ribozyme or aptazyme that has the advantage of specific gene recognition with modulation of the enzymatic activity of the gene product typically exploited by pharmaceuticals. The aptazymes of the present invention are, therefore, allosteric ribozymes in that their activity is under the allosteric control of a second portion of the ribozyme. Just as allosteric protein enzymes undergo a change in their kinetic parameters or of their enzymatic activity in response to interactions with an effector molecule, the catalytic abilities of the regulatable aptazymes may similarly be modulated by an allosteric effector(s). Thus, the present invention is directed to allosterically regulatable aptazymes that transduce molecular recognition into catalysis.

The present invention includes an allosterically regulatable aptazyme construct that is inserted into a gene of interest, e.g., a gene targeting expression vector. The regulatable aptazyme sequence provides gene specific recognition as well as modulation of the aptazyme's kinetic parameters. The kinetic parameters of the regulatable aptazyme vary in response to an allosteric effector molecule. Specifically, in the presence of the allosteric effector, the aptazyme splices itself out of the gene in response to the effector molecule to regulate expression of the gene. An important feature of the present invention is that the regulatable aptazymes disclosed herein only require recognition. rather than actual binding.

A key distinction with known systems is that the regulatory domains of the regulatable aptazymes of the present invention may bind targets, but they are engineered and selected without the necessity of knowing anything about their binding target. In the present invention it is the allosteric interaction of an effector molecule with the regulatory domain that transduces interactions into catalysis. Therefore, binding is a concomitant but secondary function of such interactions; that is, the allosteric ribozymes disclosed herein may bind the effector or the target very poorly, but upon their interaction, a synergistic effect is found that could not be detected by screening for each characteristic alone.

It is yet another embodiment of the present invention that the effector molecule does not only (or will not) produce a conformational change, but rather will add essential catalytic sites (e.g., residues) for a reaction. That is, both the effector molecule and the regulatable aptazyme contribute a portion of the active site of the ribozyme. For example, using the method of the present invention a ribozyme and an effector molecule that would be unable to bind and/or perform an enzymatic function independently, may be isolated that act synergistically. As such, an regulatable aptazyme that contributes a guanosine and an adenosine and an effector molecule that contributes a histidine from a protein effector form a synergistic effector-aptazyme complex that is regulatable based on the presence and concentration of the effector or the aptazyme. Using the methods disclosed herein it is possible to identify a chimeric effector:aptazyme (e.g., a protein:RNA complex) active site that would lead to catalysis.

The present invention also includes an aptazyme construct with a regulatable aptamer oligonucleotide sequence having a regulatory domain, such that the kinetic parameters of the aptazyme on a target gene vary in response to the interaction of an allosteric effector molecule with the regulatory domain.

The regulatable aptazyme may be used for gene expression, up regulation (increasing production of the gene product) or down regulation (decreasing the production of the gene product). The construct of one embodiment of the present invention provides a DNA oligonucleotide coding for an aptazyme domain so that the DNA can be transcribed to RNA (e.g., mRNA), where the RNA contains a self-splicing aptazyme domain that can be activated in the presence of an effector molecule. The advantages of the aptazyme technology of the present invention include the ability to continually modulate gene expression with a high degree of sensitivity without additional gene therapy interventions.

Aptazymes are more robust than allosteric protein enzymes in several ways: (1) they can be selected in vitro, which facilitates the engineering of particular constructs; (2) the levels of catalytic modulation are much greater for aptazymes than for protein enzymes; and (3) since aptazymes are nucleic acids, they can potentially interact with the genetic machinery in ways that protein molecules may not.

The regulatable aptazymes of the present invention may also be expressed inside cells. The regulatable aptazymes of the present invention that are expressed inside a cell are not only responsive to a given effector molecule, but are also able to participate in genetic regulation or responsiveness. In particular, self-splicing introns can splice themselves out of genes in response to exogenous or endogenous effector molecules.

For example, a gene can be activated or repressed in response to an exogenously introduced allosteric effector (drug) for gene therapy. In fact, al least part of the utility of the present invention is for use in the identification, isolation and enhancement of allosteric effectors and of the allosterically regulatable aptazymes with which they interact. Similarly, it is possible to activate or repress a reporter gene (e.g., luciferase) containing an engineered intron in response to an endogenous activator. In this way, luciferase-engineered intron constructs may be used to monitor intracellular levels of proteins or small molecules such as cyclic AMP. Such applications may be used for high-throughput screening. If a particular intracellular signal (e.g., the production of a tumor repressor) was desired, compound libraries are screened for pharmacophores that induce the signal (the tumor repressor), which in turn activates the intron and leads to the production of a detectable signal (e.g., expression of luciferase). Thus, the information desired is changed or morphed into the detection of glowing cells.

One important feature of using regulatable aptazymes for gene therapies is that regulated introns are a generalizable means of controlling gene expression, for any of a variety of genes, since the introns could be inserted into and be engineered to accommodate virtually any gene. Moreover, since the regulatable aptazymes may be engineered to respond to any of a variety of effectors, the characteristics of the effector (oral availability, synthetic accessibility, pharmacokinetic properties) may be chosen in advance. The drug is chosen prior to engineering the target of the drug. In part because of these extraordinary capabilities, aptazymes provide perhaps a powerful route to medically successful and practical gene therapies. Drugs may be given throughout the treatment (or lifetime) of a patient who had undergone a single initial gene therapy. By making the gene therapy regulatable, the amount of a gene product may be easily increased or decreased in different individuals at different times during the treatment by increasing or decreasing the doses of effector molecules.

The present invention includes a method for providing a regulatable aptazyme construct having an aptamer oligonucleotide sequence with a regulatory domain. A characteristic of the regulatable aptazyme construct of the present method is that the kinetic parameters of the aptazyme vary in response to an effector molecule. In particular, the kinetic parameters of the aptazyme on a target gene vary in response to the interaction of an allosteric effector molecule with the regulatory domain. For example, the aptazyme splices itself out of a gene in response to the effector molecule interacting with the regulatory domain of the aptazyme to regulate expression of the target gene.

The present method also includes transforming a cell with the regulatable aptazyme construct so that the aptazyme construct is inserted into a gene of interest. An effector molecule is provided to activate the aptazyme so that administering to the cell an effective amount of the allosteric effector molecule induces the aptazyme to splice itself out of the gene to regulate expression of the, gene.

The method of the present invention contemplates that the aptazyme construct may be within a plasmid. The method then further includes transforming the cell with the aptazyme construct containing plasmid. The method of the present invention also includes ligating the regulatable aptazyme construct into a vector and transforming the cell with the vector. Additionally, the method of the present invention contemplates that the regulatable aptazyme construct may be amplified by polymerase chain reaction. Finally, the method contemplates that the regulatable aptamer oligonucleotide sequence of the construct may include RNA nucleotides, so that the method further includes reverse transcription of the RNA using reverse transcriptase to produce an DNA aptamer complementary to the RNA template.

The invention also includes the automation of in vitro selection, and a mechanized system that executes both common and modified in vitro selection procedures. Automation facilitates the execution of this procedure, accomplishing in hours-to-days what once necessitated weeks-to-months. Additionally, the mechanized system attends to other technical obstacles not addressed in ‘common’ in vitro selection procedure (e.g, specialized robotic manipulation to avoid cross-contamination).

The automation methods are generalizable to a number of different types of selections, including selections with DNA or modified RNA, selections for ribozymes and selections for phage-displayed or cell-surface displayed proteins.

Automating selection greatly diminishes human error in the actual pipetting and biological manipulations. While programming the robot is often not a trivial task, and can be time consuming, automated selection is far faster and more efficient than manual selection. The scientist's time is thus put to better use preparing samples and analyzing data, rather than performing the actual selection. Additionally, automated selection may include real-time monitoring methods (e.g., molecular beacons, TaqMan®) into the selection procedure and software that can make intelligent decisions based on real-time monitoring.

Aptazymes, or allosterically activated ribozymes, have been developed that are activated cyclic nucleotide monophosphates as well as other small molecules like theophylline. In addition to aptazymes activated by small molecules, the present invention also includes allosteric ribozymes that are activated by protein cofactors. Indeed, there are natural ribozymes that are extremely dependent on proteins for their activity.

One embodiment of the present invention involves the in vitro selection of ribozymes that are allosterically activated by proteins. A selection scheme for ribozymes that are dependent on protein cofactors has been developed. A novel class of nucleoprotein enzymes has been identified where the enzymes are even more dependent on Cytl8 for their activity than is the natural Group I intron. Aptazymes may be allosterically regulated by any of,a vast number of proteins.

Other methods for aptazyme development using small molecule ligands have proven successful. In particular, it has been possible to add aptamer moieties to ribozymes, without selection, and achieve activation in the presence of ligand (like ATP or theophylline). However, this has proved difficult in the case of protein ligands. One of the unique features of the present selection protocol relative to others that have previously been published is that the present invention randomizes not only a domain that is pendant on the catalytic core, but a portion of the catalytic core itself. Thus, the selection for protein-dependence not only yields species which bind to ancillary regions of the ribozyme, but that likely help organize the catalytic core of the ribozyme.

It should be noted that the method is not limited to RNA pools, but could also encompass DNA pools or modified RNA pools. The method is not limited to ligases, but could also encompass other ribozyme classes. The method is not limited to protein-induced conformational changes, but could also take into account ‘chimeric’ catalysts in which residues essential for chemical reactivity were provided by both the nucleic acid and the protein, in concert.

This invention allows the selection of protein-dependent aptazymes, which are extremely novel reagents that can be useful in a variety of applications. For example, protein-dependent aptazymes can be used (1) in substrates for the acquisition of data about whole proteomes, (2) as in vitro diagnostic reagents to detect proteins specific to disease states, such as prostate-specific antigen (PSA) or viral proteins, (3) as sentinels for the detection of biological warfare agents, or (4) as regulatory elements in gene therapies, as described herein.

Initially, many protein targets may prove refractive to selection. However, many derivatives of the base method can be developed, to deal with novel targets or target classes.

It has been shown that ribozyme catalysis can be modulated by allosteric effectors. In yet another embodiment of the present invention, these allosteric ribozymes, also referred to as aptazymes, are displated in arrays to be used for monitoring the presence of various molecules, be they inorganic or organic (e.g., metabolites or proteins).

For example, aptazymes are anchored to a substrate, such as wells in a multi-well plate, and different ribozyme ligases are covalently immobilized on beads in the wells. Mixtures of analytes and fluorescently tagged substrates are added to each well. Where cognate effectors are present, the aptazymes will covalently attach the fluoreschent tags to themselves. Where aptazymes have not been activated by effectors, the tagged substrates are washed out of the well. After reaction and washing, the presence and amounts of co-immobilized fluorescent tags are indicative of amounts of ligands that were present during the reaction.

In this embodiment of the invention, the reporter may be a fluorescent tag, but it may also be an enzyme, a magnetic particle, or any number of detectible particles. Additionally, the ribozymes could be immobilized on beads, but they could also be directly attached to a solid support via covalent bonds.

One advantage of this embodiment is that covalent immobilization of reporters (as opposed to non covalent immobilization, as in ELISA™ assays) allows stringent wash steps to be employed. Additionally, ribozyme ligases have the unique property of being able to transduce effectors into templates that can be amplified, affording an additional boost in signal prior to detection.

Nucleic acids are generally less robust than antibodies. However, modified nucleotides may be introduced into the aptazymes that substantially stabilize them from degradation in environments such as sera or urine. Similarly, antibodies generally have higher affinities for analytes than do aptamers, and be inference aptazymes. However, the analytical methods of the present invention do not rely on binding per se, but only on transient interactions. The present invention requires mere recognition rather that actual binding, providing a potential advantage of apatzymes over antibodies. That is, even low affinities are sufficient for activation and subsequent detection, especially if individual immobilized aptazymes are examined (i.e., by ligand-dependent immobilization of a quantum dot).

### BRIEF DESCRIPTION OF THE DRAWINGS

For a more complete understanding of the features and advantages of the present invention, reference is now made to the detailed description of the invention along with the accompanying figures in which corresponding numerals in different figures refer to corresponding parts and in which:

FIG. 1 is a depiction of the secondary structure of the Group 1 theophylline-dependent (td) intron of bacteriophage T4 (wild type);

FIG. 2a is a photograph of a gel showing activation of the GpITh1P6.131 aptamer construct, together with a graphical representation of the gel, of one embodiment of the present invention;

FIG. 2b is a photograph of a gel showing activation of GpITh2P6.133 aptamer construct, together with a graphical representation of the gel of one embodiment of the present invention.

FIG. 3 is a schematic depiction of an in vivo assay system for group I introns of one embodiment of the present invention.

FIG. 4a depicts a portion of the P6 region of the Group I ribozyme joined to the anti-theophylline aptamer by a short randomized region to generate a pool of aptazymes of the present invention.

FIG. 4b is a schematic depiction of a selection protocol for the Group I P6 Aptazyme Pool of FIG. 4a.

FIG. 5 is a diagram of one embodiment of the present invention depicting exogenous or endogenous activation of Group I intron splicing;

FIG. 6 is a diagram of another embodiment of the present invention depicting a strategy for screening libraries of exogenous activators;

FIG. 7 is a diagram of an alternative embodiment of the present invention for screening libraries of exogenous activators;

FIG. 8 is a diagram of yet another alternative embodiment of the present invention for screening libraries of exogenous activators;

FIG. 9 is a diagram of an embodiment of the present invention for screening for endogenous activators;

FIG. 10 is a diagram of an alternative to the embodiment of FIG. 9 of the present invention to screen for endogenous activators;

FIG. 11 is a diagram of another embodiment of the present invention to screen for compounds that perturb cellular metabolism;

FIG. 12 is a diagram of a further embodiment of the present invention that provides a non-invasive readout of metabolic states;

FIG. 13 is a diagram of yet a further embodiment of the present invention wherein endogenous suppressors provide a non-invasive readout of multiple metabolic states;

FIG. 14 is a schematic depiction of an example of a worksurface for automatic selection procedures of one embodiment of the invention;

FIG. 15a is an illustration of the LI ligase aptazyme construct of one embodiment of the present invention;

FIG. 15b is an illustration of a modified LI ligase aptazyme construct of FIG. 15a of the present invention;

FIG. 15c is a schematic diagram of a selection protocol of one embodiment of the present invention; and

FIG. 16 is a schematic diagram of a method to anchor an aptazyme construct of the present invention to a solid support in one embodiment of the present invention.

### DETAILED DESCRIPTION OF THE INVENTION

While the making and using of various embodiments of the present invention are discussed in detail below, it should be appreciated that the present invention provides many applicable inventive concepts that may be embodied in a wide variety of specific contexts. The specific embodiments discussed herein are merely illustrative of specific ways to make and use the invention and do not delimit the scope of the invention.

Definitions

As used herein, the term “regulatable aptazyme” means an allosteric ribozyme. The kinetic parameters of the ribozyme may be varied in response to the amount of an allosteric effector molecule. Just as allosteric protein enzymes undergo a change in their kinetic parameters or of their enzymatic activity in response to interactions with an effector molecule, the catalytic abilities of regulatable aptazymes can similarly be modulated by effectors. Regulatable aptazymes transduce molecular recognition into catalysis upon interaction with an allosteric effector molecule that binds an effector portion of the regulatable aptazyme. Specifically, in the presence of the effector, the aptazyme splices itself out of a gene in response to the effector molecule to regulate expression of the gene.

As used herein, the term “aptamer” refers to an oligonucleotide having aptazyme activity.

As used herein, the term “allosteric effector” or “allosteric effector molecule” means a substance that allosterically changes the kinetic parameters or catalytic activity of an aptazyme, and in particular a substance that activates self-splicing of an aptazyme.

As used herein, the term “kinetic parameters” refers to catalytic activity. Changes in the kinetic parameters of a catalytic ribozyme produce changes in the catalytic activity of the ribozyme such as a change in the rate of reaction or a change in substrate specificity. For example, self-splicing of an aptazyme out of a gene environment may result from a change in the kinetic parameters of the aptazyme.

As used herein, the term “catalytic” or “catalytic activity” refers to the ability of a substance to affect a change in itself or of a substrate under permissive conditions.

As used herein, the term “protein-protein complex” or “protein complex” refers to an association of more than one protein. The proteins of the complex may be associated by a variety of means, or by any combination of means, including but not limited to functional, stereochemical, conformational, biochemical, or electrostatic association. It is intended that the term encompass associations of any number of proteins.

As used herein the terms “protein”, “polypeptide” or “peptide” refer to compounds comprising amino acids joined via peptide bonds and are used interchangeably.

As used herein, the term “endogenous” refers to a substance the source of which is from within a cell. Endogenous substances are produced by the metabolic activity of a cell. Endogenous substances, however, may nevertheless be produced as a result of manipulation of cellular metabolism to, for example, make the cell express the gene encoding the substance.

As used herein, the term “exogenous” refers to a substance the source of which is external to a cell. An exogenous substance may nevertheless be internalized by a cell by any one of a variety of metabolic or induced means known to those skilled in the art.

As used herein, the term “gene” means the coding region of a deoxyribonucleotide sequence encoding the amino acid sequence of a protein. The term includes sequences located adjacent to the coding region on both the 5′, and 3′, ends such that the deoxyribonucleotide sequence corresponds to the length of the full-length mRNA for the protein. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed, excised or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the MRNA transcript). The 5′ flanking region may contain regulatory sequences such as promoters and enhancers which control or influence the transcription of the gene. The 3′ flanking region may contain sequences which direct the termination of transcription, post-transcriptional cleavage and polyadenylation.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand.

The term “gene of interest” as used here refers to a gene, the function and/or expression of which is desired to be investigated, or the expression of which is desired to be regulated, by the present invention. In the present disclosure, the td gene of the T4 bacteriophage is an example of a gene of interest and is described herein to illustrate the invention. The present invention may be useful in regard to any gene of any organism, whether of a prokaryotic or eukaryotic organism.

The term “hybridize” as used herein, refers to any process by which a strand of nucleic acid binds with a complementary strand through base pairing. Hybridization and the strength of hybridization (i.e., the strength of the association between the nucleic acid strands) is impacted by such factors as the degree of complementary between the nucleic acids, stringency of the conditions involved, the melting temperature of the formed hybrid, and the G:C (or U:C for RNA) ratio within the nucleic acids.

The terms “complementary” or “complementarity” as used herein, refer to the natural binding of polynucleotides under permissive salt and temperature conditions by base-pairing. For example, for the sequence “A-G-T” binds to the complementary sequence “T-C-A”. Complementarity between two single-stranded molecules may be partial, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands.

The term “homology,” as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence. When used in reference to a single-stranded nucleic acid sequence, the term “substantially homologous” refers to any probe which can hybridize (i.e., it is the complement of) the single-stranded nucleic acid sequence under conditions of low stringency as described.

As known in the art, numerous equivalent conditions may be employed to comprise either low or high stringency conditions. Factors such as the length and nature (DNA, RNA, base composition) of the sequence, nature of the target (DNA, RNA, base composition, presence in solution or immobilization, etc.), and the concentration of the salts and other components (e.g., the presence or absence of formamide, dextran sulfate and/or polyethylene glycol) are considered and the hybridization solution may be varied to generate conditions of either low or high stringency different from, but equivalent to, the above listed conditions.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

Low stringency conditions comprise conditions equivalent to binding or hybridization at 42° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5× Denhardt's reagent (50× Denhardt's contains per 500 ml: 5 g Ficoll (Type 400, Pharmacia), 5 g BSA [Fraction V; Sigma]) and 100 μg/ml denatured salmon sperm DNA) followed by washing in a solution comprising 5×SSPE, 01% SDS at 42° C. when a probe of about 500 nucleotides in length is employed.

Numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions which promote hybridization under conditions of high stringency (e.g., increasing the temperature of the hybridization and/or wash steps, the use of formamide in the hybridization solution, etc.).

The term “antisense,” as used herein, refers to nucleotide sequences that are complementary to a specific DNA or RNA sequence. The term “antisense strand” is used in reference to a nucleic acid strand that is complementary to tile “sense” strand. Antisense molecules may be produced by any method, including synthesis by ligating the gene(s) of interest in a reverse orientation to a viral promoter which permits the synthesis of a complementary strand. Once introduced into a cell, the transcribed strand combines with natural sequences produced by the cell to form duplexes. These duplexes then block either the further transcription or translation. In this manner, mutant phenotypes may also be generated. The designation “negative” is sometimes used in reference to the antisense strand, and “positive” is sometimes used in reference to the sense strand.

The term also is used in reference to RNA sequences that are complementary to a specific RNA sequence (e.g., mRNA) Included within this definition are antisense RNA (“asRNA”) molecules involved in genetic regulation by bacteria. Antisense RNA may be produced by any method, including synthesis by splicing the gene(s) of interest in a reverse orientation to a viral promoter which permits the synthesis of a coding strand. Once introduced into an embryo, this transcribed strand combines with natural mRNA produced by the embryo to form duplexes. These duplexes then block either the further transcription of the mRNA or its translation. In this manner, mutant phenotypes may be generated. The term “antisense strand” is used in reference to a nucleic acid strand that is complementary to the “sense” strand. The designation. (−) (i.e., “negative”) is sometimes used in reference to the antisense strand with the designation (+) sometimes used in reference to the sense (i.e., “positive”) strand.

A gene may produce multiple RNA species which are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA I wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

“Transformation,” as defined herein, describes a process by which exogenous. DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. The term “transfection” as used herein refers to the introduction of foreign DNA into eukaryotic cells.

Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “selectable marker” refers to the use of a gene that encodes an enzymatic activity and which confers the ability to grow in medium lacking what would otherwise be an essential nutrient (e.g., the HIS3 gene in yeast cells); in addition, a selectable marker may confer resistance to an antibiotic or drug upon the cell in which the selectable marker is expressed. A review of the use of selectable markers in mammalian cell lines is provided in Sambrook, J. et. a!., Molecular Cloning: A Laboratory Manual, 2nd ed., Cold Spring Harbor Laboratory Press, New York (1989) pp.16.9-16.15.

As used herein, the term “reporter gene” refers to a gene that is expressed in a cell upon satisfaction of one or more contingencies and which, upon expression, confers a detectable phenotype to the cell to indicate that the contingencies for expression have been satisfied. For example, the gene for Luciferase confers a luminescent phenotype to a cell when the gene is expressed inside the cell. In the present invention, the gene for Luciferase may be used as a reporter gene such that the gene is only expressed upon the splicing out of an intron in response to an effector. Those cells in which the effector activates splicing of. the intron will express Luciferase and will glow.

As used herein, the term “vector” is used in reference to nucleic acid molecules that transfer DNA segment(s) from one cell to another. The term “vehicle” is sometimes used interchangeably with “vector.”

The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “amplify”, when used in reference to nucleic acids refers to the production of a large number of copies of a nucleic acid sequence by any method known in the art. Amplification is a special case of nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic ′d strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted oat from other nucleic acid sequences. A “segment” is defined as a region of nucleic acid within the target sequence.

As used herein, the term “polymerase chain reaction” (“PCR”) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence.

To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

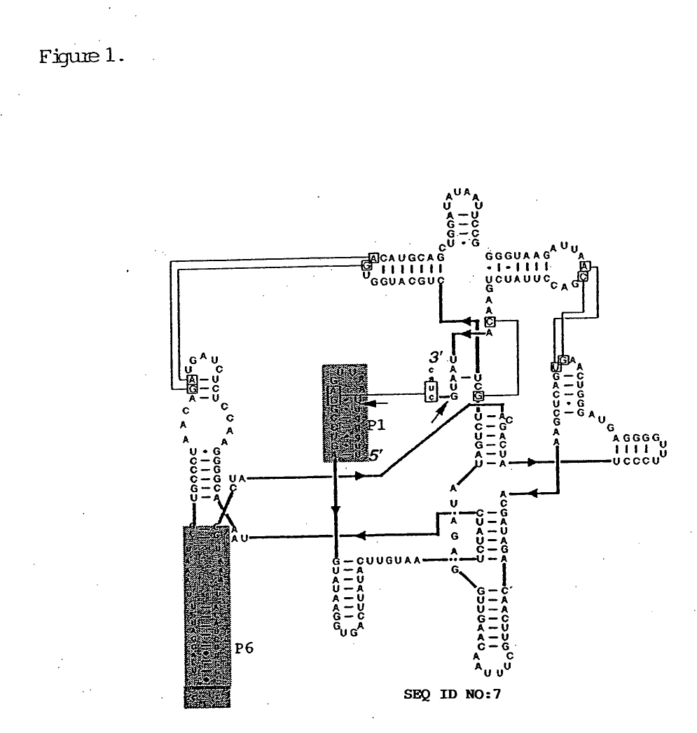

The invention is now described in detail with the use of the td intron from T4 bacteriophage (FIG. 1) for illustrative purposes. The description is not intended to limit the scope of the invention or the claims appended hereto.

FIG. 1 depicts the secondary structure of the td intron from bacteriophage T4 (GenBank # M12742), wherein “td” means theophylline-dependent. The td intron was selected to illustrate the present invention because, among other things, mutational analysis has identified regions of this intron that can be engineered and modified. See Salvo, et al., Deletion-tolerance and trans-splicing of the bacteriophage T4 intron. Analysis of the P6-L6a region. J. Mol. Biol. 211, 537-549 (1990) and Salvo et al., The P2 element of the td intron is dispensable despite its normal role in splicing. J. Mol. Biol. 267, 2845-2848 (1992). Thus, aptamer domains or pools may be engineered into the T4 intron.

The intron has been adapted to a prokaryotic in vivo screening protocol. M. Belfort, et al., Genetic delineation of functional components of the group I intron in the phage T4 td gene. Cold Spring Harb Symp Quant Biol 52, 181-92 (1987). The present invention improves the Belfort, et al., protocol to assay drug dependence in vivo. The protocol may also be used to screen and select Group I aptazymes in vivo.

A final advantage of the T4 intron is that the intron can be rendered protein-dependent. C.A. Myers, et al., A tyrosyl-tRNA synthetase suppresses structural defects in the two major helical domains of the group I intron catalytic core. J. Mol. Biol. 262 87--104 (1996). An anti-theophilline aptamer has been described by R. D. Jenison, et al., High-resolution molecular discrimination by RNA. Science 263, 1425-1429 (1994).

In the present invention, the anti-theophylline aptamer was mounted in two locations in the td intron, shown by the shaded portions of FIG. 1. One location was at the termini of P1 and the other location was within P6. The P1 constructs may enable ligand-dependent conformational changes that alter the conformation or register of the U:G base pair which is critical for splicing. The P6 region was selected because mutational analysis. indicated that deletion of the P6 stem destabilizes the intron.

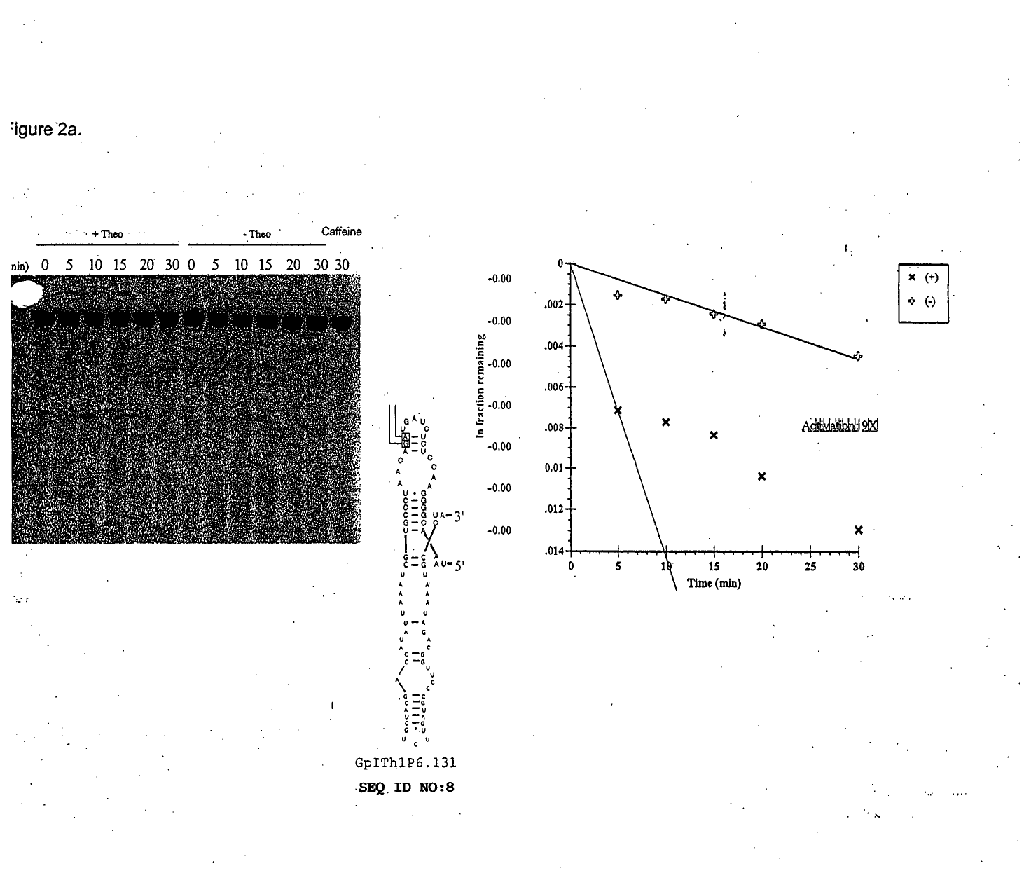

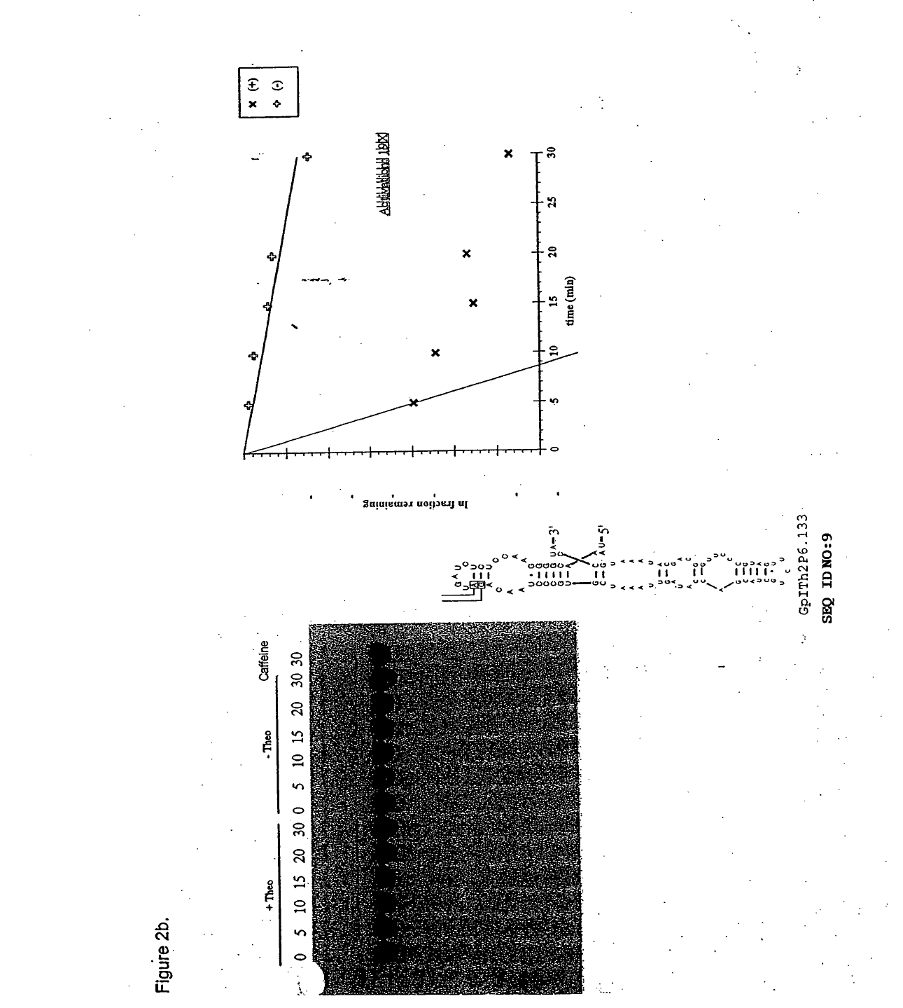

Referring now to FIGS. 2a and 2b, in the present invention P6 constructs were made so that Group I splicing was activated by the presence of theophilline in the range of approximately 9 to 19 fold over constructs grown in the absence of theophilline, as described in the examples below:

The following examples illustrate the present invention in the td gene system of T4. For a full understanding of the examples, refer to FIGS. 2a and 2b. The examples are provide for illustrative purposes and do not limit the scope of the present invention or the scope of the appended claims.

### EXAMPLE 1

### GpITh1P6

### Engineering of Group I Aptazymes

The first example illustrates how to make an aptazyme construct and demonstrates self-splicing of the aptazyme out of a gene in response to an effector molecule.

Construction of a Regulatable Aptazyme Oligos GpIWt3.129: 5′-TAA TCT TAC CCC GGA ATT ATA TCC AGC TGC ATG TCA CCA TGC AGA GCA GAC TAT ATC TCC AAC TTG TTA AAG CAA GTT GTC TAT CGT TTC GAG TCA CTT GAC CCT ACT CCC CAA AGG GAT AGT CGT TAG-3′ (SEQ ID NO: 1) and GpIThlP6.131: 5′-GCC TGA GTA TAA GGT GAC TTA TAC TTG TAA TCT ATC TAA ACG GGG AAC CTC TCT AGT AGA CAA TCC CGT GCT AAA TTA TAC CAG CAT CGT CTT GAT GCC CTT GGC AGA TAA ATG CCT AAC GAC TAT CCC TT-3′ (SEQ ID NO: 2) were annealed and extended in a 30 μl reaction containing 100 pmoles of each oligo, 250 mM Tris-HCl (pH 8.3), 40 mM MgCl_{2,} 250 mM NaCl, 5 mM DTT, 0.2 mM each dNTP, 45 units of AMV reverse transcriptase (RT: Amersham Pharmacia Biotech, Inc., Piscataway, N.J.) at 370 ° C. for 30 minutes. The extension reaction was diluted 1 to 50 in H₂O.

A PCR reaction containing 1 μl of the extension dilution, 500 mM KCl, 100 mM Tris-HCl, (pH 9.0), 1% Triton® x-100, 15 mM MgCl_{2,} 0.4 μM of GpIWt1.75: 5′-GAT AAT ACG ACT CAC TAT AGG GAT CAA CGC TCA GTA GAT GTT TTC TTG GGT TAA TTG AGG CCT GAG TAT AAG GTG-3′ (SEQ ID NO:3), 0.4 μM of GpIWt4.89: 5′-CTT AGC TAC AAT ATG AAC TAA CGT AGC ATA TGA CGC AAT ATT AAA CGG TAG CAT TAT GTT CAG ATA AGG TCG TTA ATC TTA CCC CGG AA-3′ (SEQ ID NO:4), 0.2 mM each dNTP and 1.5 units of Taq polymerase (Promega, Madison, Wis.) was thermocycled 20 times under the following regime: 94° C. for 30 seconds, 45° C. for 30 seconds, 720° C. for 1 minute. The PCR reaction was precipitated in the presence of 0.2 M NaCl and 2.5 volumes of ethanol and then quantitated by comparison with a molecular weight standard using agarose gel electrophoresis.

The aptazyme construct was transcribed in a 10 μl high yield transcription reaction (AmpliScribe from Epicentre, Madison, Wis. The reaction contained 500 ng PCR product, 3.3 pmoles of p³² [α-32 P]UTP (3000 Camel), 1× AmpliScribe transcription buffer, 10 mM DTT, 7.5 mM each NTP, and 1 μl AmpliScribe T7 polymerase mix. The transcription reaction was incubated at 37° C. for 2 hours. One unit of RNase free-DNase was added and the reaction returned to 37° C. for 30 minutes. The transcription was then purified on a 6% denaturing polyacrylamide gel to separate the full length RNA from incomplete transcripts and spliced products, eluted and quantitated spectrophotometrically.

In Vitro Assay The RNA (4 pmoles/12 μl H₂O) was heated to 94° C. for 1 minute then cooled to 37° C. over 2 minutes in a thermocycler. The RNA was divided into 2 splicing reactions (9 μl each) containing 100 mM Tris-HCl (pH 7.45), 500 mM KCl and 15 mM MgCl₂, plus or minus theophylline (2 mM). The reactions were immediately placed on ice for 30 minutes. GTP (1 mM) was added to the reactions (final volume of 10 μl) and the reactions were incubated at 37° C. for 2 hours.

The reactions were terminated by the addition of stop dye (10 μl) (95% formamide, 20 mM EDTA, 0.5% xylene cyanol, and 0.5% bromophenol blue). The reactions were heated to 70° C. for 3 minutes and 10 μl was electrophoresed on a 6% denaturing polyacrylamide gel. The gel was dried, exposed to a phosphor screen and analyzed using a Molecular Dynamics Phosphorimager (Sunnyvale, Calif.).

Activation was determined from the amount of circular intron in each reaction. Circularized introns migrate slower than linear RNA and can be seen as the bands above the dark bands of linear RNA in the +Theo lanes of the gels of FIGS. 2a and 2b.

In Vivo Screening of Group I Aptazymes

The aptazyme constructs as well as the wild type and a negative control were ligated into a vector that contains the T4 td intron with Eco RI and Spe I flanking the P6 region, transformed and minipreped. The plasmids were then transformed into C600:Thy A Kan^{R} cells (cells lacking thymidine synthetase).

Individual colonies were picked and grown in rich media overnight. Theophylline (1 μl: 6.6 mM) or H₂O (1 μl) was added to 2 μl of the overnight growth and was spotted on either minimal media plates, or minimal media plates with thymine.

### EXAMPLE 2

### GpIP6Thpool

### In Vitro Selection of Group I Aptazymes

Example 2 illustrates how to generate a population of aptazymes so that there is variation in the nucleotide sequence of the aptamers. This example also illustrates how to select for phenotypes that are responsive to an effector molecule from among that population of aptazymes.

Construction of Pool

The construction of the pool was similar to the construction of the individual engineered aptazyme constructs. Oligos GpIWt3.129 and GpIThP6pool: 5′-GCC TGA GTA TAA GGT GAC TTA TAC TAG TAA TCT ATC TAA ACG GGG AAC CTC TCT AGT AGA CAA TCC CGT GCT AAA TN(1-4)A TAC CAG CAT CGT CTT GAT GCC CTT GGC AGN(1-4) TAA ATG CCT AAC GAC TAT CCC TT-3′ (SEQ ID NO:5) were extended in the same manner as above. The extension reaction was diluted and used as template for a PCR reaction. The PCR reaction was similar to the reaction described with the following exceptions: the volume was doubled and GpIWt4.89 was replaced with GplMutG.101: 5′-CTT AGC TAC AAT ATG AAC TAA CGT AGC ATA TGA CGC AAT ATT AAA CGG TAG TAT TAT GTT CAG ATA AGG TCG TTA ATC TTA CCC CGG AAT TCT ATC CAG CT-3′ (SEQ ID NO:6) in which there is an G to A mutation at the terminal residue of the intron. The pool had a diversity of 1.16×10⁵ molecules. RNA was made as described above.

In Vitro Negative Selection

The RNA (10 pmoles/70 μl H₂O) was heated to 94° C. for 1 minute then cooled to 37° C. over 2 minutes in a thermocycler. The splicing reaction (90 μl) contained 100 mM Tris-HCl (pH 7.45), 500 mM KCl and 15 mM MgCl₂. The reaction was immediately placed on ice for 30 minutes. GTP (1 mM) was added to the reaction (final volume of 100 μl) and the reaction was incubated at 37° C. for 20 hours. The reaction was terminated by the addition 20 mM EDTA and precipitated in the presence of 0.2 M NaCl and 2.5 volumes of ethanol. The reaction was resuspended in 10 μl H₂O and 10 μl stop dye and heated to 70° C. for 3 minutes and was electrophoresed on a 6% denaturing polyacrylamide gel with Century™ Marker ladder (Ambion, Austin, Tex.). The gel was exposed to a phosphor screen and analyzed. The unreacted RNA was isolated from the gel, precipitated and resuspended in 10 μl of H₂O.

Positive Selection

The RNA (5 μl of negative selection) was heated to 94° C. for 1 minute then cooled to 37° C. over 2 minutes in a thermocycler. The positive splicing reaction (45 μl) contained 100 mM Tris-HCl (pH 7.45), 500 mM KCl, 15 mM MgCl₂ and 1 mM theophylline. The reaction was immediately placed on ice for 30 minutes. GTP (1 mM) was added to the reaction (final volume of 50 μl) and the reaction was incubated at 37° C. C. for 1 hour. The reaction was terminated by the addition of stop dye, heated to 70° C. for 3 minutes and was electrophoresed on a 6% denaturing polyacrylamide gel with Century™ Marker ladder. The gel was exposed to a phosphor screen and analyzed. The band corresponding to the linear intron was isolated from the gel and precipitated and resuspended in 20 μl H₂O.

Amplification and Transcription

The RNA was reverse transcribed in a reaction containing 250 mM Tris-HCl (pH 8.3), 375 mM KCl, 15 mM, MgCl₂, 0.1 M DTT, 0.4 mM of each dNTP 2 μM GpIMutG.101 and 400 units of SuperScript II reverse transcriptase (Gibco BRL, Rockville, Md.). The cDNA was then PCR amplified, transcribed and gel purified as described above.

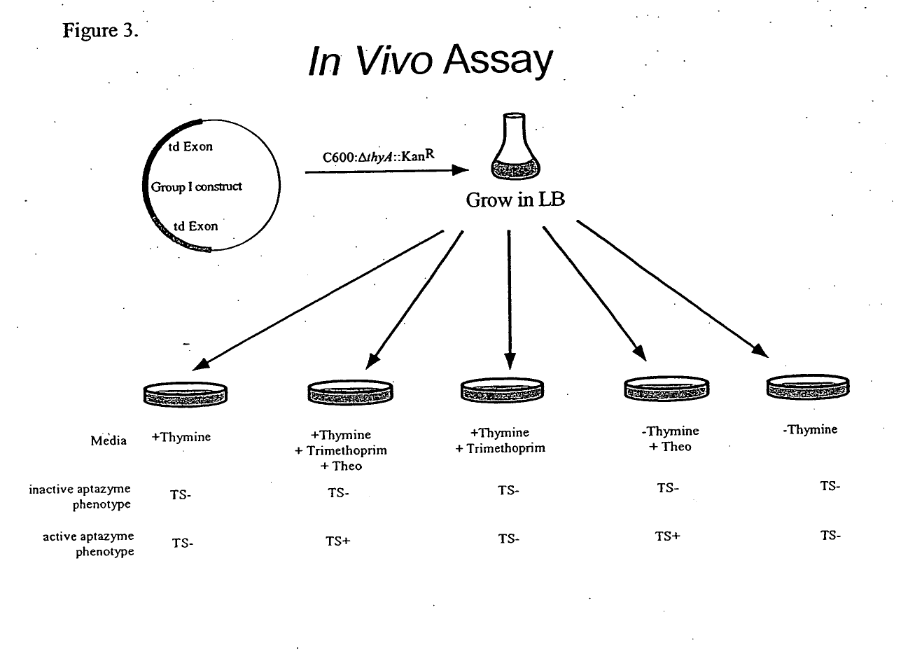

FIG. 3 depicts an in vivo assay system for Group I introns of the present invention. The td intron normally sits within the td gene for thymidylate synthase (TS) in phage T4. A ThyA E. coli host that lacks celluar TS is unable to grow in the absence of exogenous thymine or thymidine (−Thy). The cloned td gene can complement the ThyA cells and grow on −Thy media. Conversely, cells that lack TS have a selective. advantage on media containing thymidine and trimethoprim. Therefore, cells harboring theophylline-responsive Group I aptazymes grow better in the presence of theophilline and the absence of thymidine. In contrast,.the same cells grow better in the absence of theophylline and the presence of thymidine and trimethoprim.

This strategy provides both a positive in vivo screen and selection for theophylline-dependent activation and a negative in vivo screen and selection for theophylline-absent repression. The assay system of FIG. 3 was used in Example 1, above, for the in vivo screening of Group I aptazymes in a specific embodiment of the present invention.

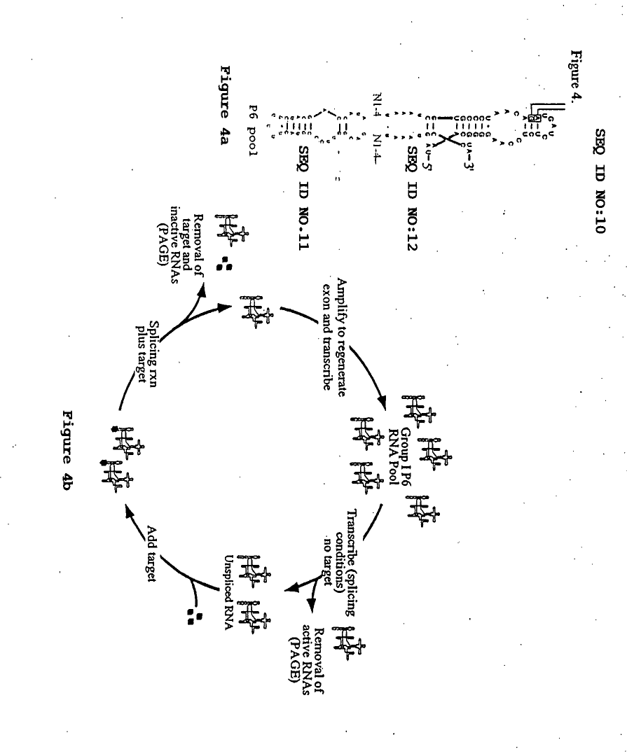

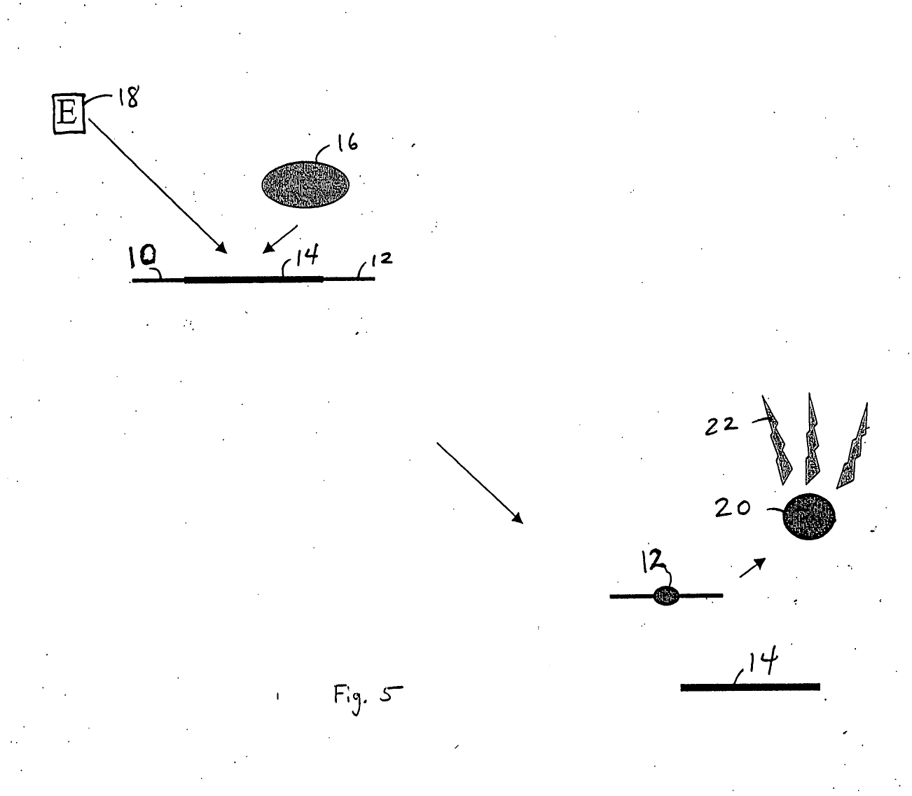

FIG. 4a depicts the critical residues of the P6 region of the Group I ribozyme joined to the anti-theophylline aptamer by a short randomized region to generate a pool of aptazymes of the present invention. The residues shown in bold in FIG. 4a are the P6 critical residues, and the faded residues shown in FIG. 4a are the anti-theophylline aptamer. The randomized regions are designated in FIG. 4a as “N1-4”. Approximately 40 random sequence residues are introduced into the N1-4 region of the construct to synthesize a pool of aptazymes, referred to herein as a communication module pool.

FIG. 4b shows a selection protocol for the Group I P6 Aptazyme Pool of FIG. 4a. Positive and negative selections are made in vitro to select Group I aptazymes that are dependent on theophylline. The selections are described above in Example 2 for a specific embodiment of the present invention. In vivo screens and selections are used to select Group I aptazymes that exhibit strong theophylline-dependence. The selected aptazymes are mixed at various ratios with mutant Group I ribozymes that splice at a low but continuous level to determine the level at which aptazymes can be selected against background. A communication module pool can be transformed with the selected aptazymes to determine whether the same modules that function in vitro are also functional in vivo. Finally, the best theophylline-dependent Group I aptazymes that have been derived by any of the methods described herein may undergo further selection by partially randomizing their sequences and selecting for improved in vivo performance.

Strategies similar to those depicted in FIGS. 4a and 4b may be used to develop aptazymes and aptamers dependent on any desired effector molecule. See generally G. A. Soukup, et al., Engineering precision RNA molecular switches. Proc. Natl. Acad. Sci. U.S.A. 96, 3584-3589 (1999) and M. Koizumi, et al., Allosteric selection of ribozymes that respond to the second messengers cGMP and cAMP. Nature Struct. Biol. 6, 1062-1071 (1999). Positive and negative in vitro selection such as depicted in FIG. 4b are described above in Example 2 for a specific embodiment of the present invention.

From 10⁶ to 10¹⁰ variants can be efficiently transformed as described herein, sufficient to encompass most variants in the populations discussed so far. This efficiency of transformation, however, is likely to be insufficient to encompass a significant fraction of a completely random pool. Nonetheless, sequences have been selected from completely random expressed pools that can protect bacteria from bacteriophage infection.

The optimization strategies described herein yield Group I aptazymes that are highly dependent on small molecule effectors. Since the rules that govern Group I intron splicing in different gene contexts are well known to those skilled in the art, the skilled artisan can remove Group I aptazymes from the td gene and modularly insert them into other genes. Should the efficiency or effector-dependence of intron splicing be compromised in the new gene, the intron can be reaccommodated to its new genetic environment by fusing td or another selectable marker to the interrupted gene of interest and selecting for an effector-dependent phenotype.

To the extent that Group I aptazymes are self-sufficient, they should also function in eukaryotic cells, including human cells. However, to the extent that the architecture of eukaryotic cells is significantly different from the cytoplasm of bacteria, the efficiency or effector-dependence of intron splicing may suffer on aptazyme transfer between different species of organisms. Selecting for effector-dependence, but now in eukaryotic cells, may be necessary to obtain satisfactory efficiency and effector-dependence. Selection in eukaryotic systems may be performed by fusing the gene of interest to a reporter gene such as GFP or luciferase. Variants of the aptazyme that promote effector-dependent protein production may then be selected using a FACS. 10⁶ to 10⁸ variants may be screened by this procedure, a range comparable to the bacterial system previously described.

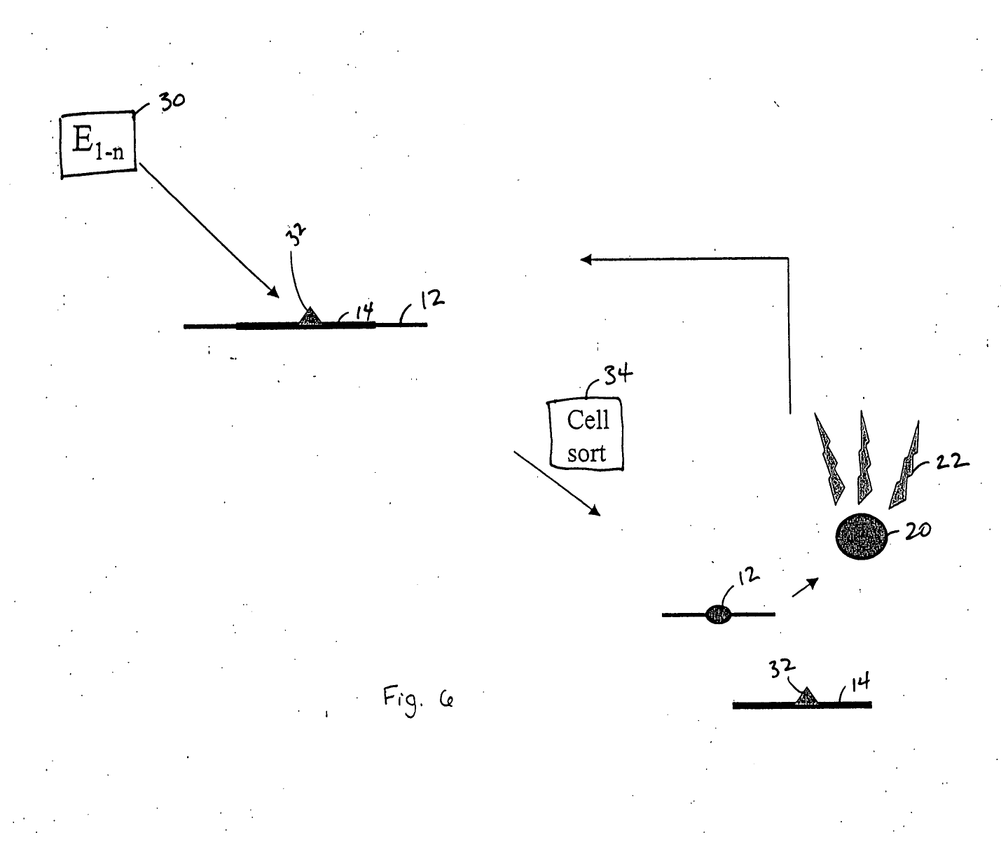

FIG. 5 is a diagrammatic representation of one embodiment of the exogenous or endogenous activation of Group I intron splicing is depicted. A gene of interest 10 is fused to a reporter gene 12 such as luciferase or beta-galactosidase, which also contains the group I intron (td) 14. Splicing-out of the Group I intron is induced by an endogenous effector molecule 16, which may be a protein, e.g., Cytl8. Alternatively, splicing-out of the Group I intron may be induced by an exogenous effector molecule 18. Activation of the aptazyme and auto-excision of the intron results in expression of the reporter gene encoded protein 20 that is detect by, e.g., fluorescence 22 or any other desired detectable reaction. The use of a reporter gene 12 of this embodiment may be suitable for use in eukaryotic systems.

FIG. 6 is a diagram of another embodiment of the present invention. Libraries of candidate exogenous activators 30 can be generated from a randomized aptazyme pool indicated by random loop. E₁₋ₙ. As in the embodiment of FIG. 5, a reporter gene 12 is expressed in cells where the exogenous activator 30 activates the aptazyme to release the intron, which may contain a random loop 32, from the gene. In this embodiment, the reaction occurs within cells which are then sorted 34 based on a chromogenic reaction or emission 22, or may even be isolated by, e.g, statistical cell separation cloning. As will be known to those of skill in the art of enzymatic oligonucleotides any number of current and future effector molecule libraries may be used with the present invention.

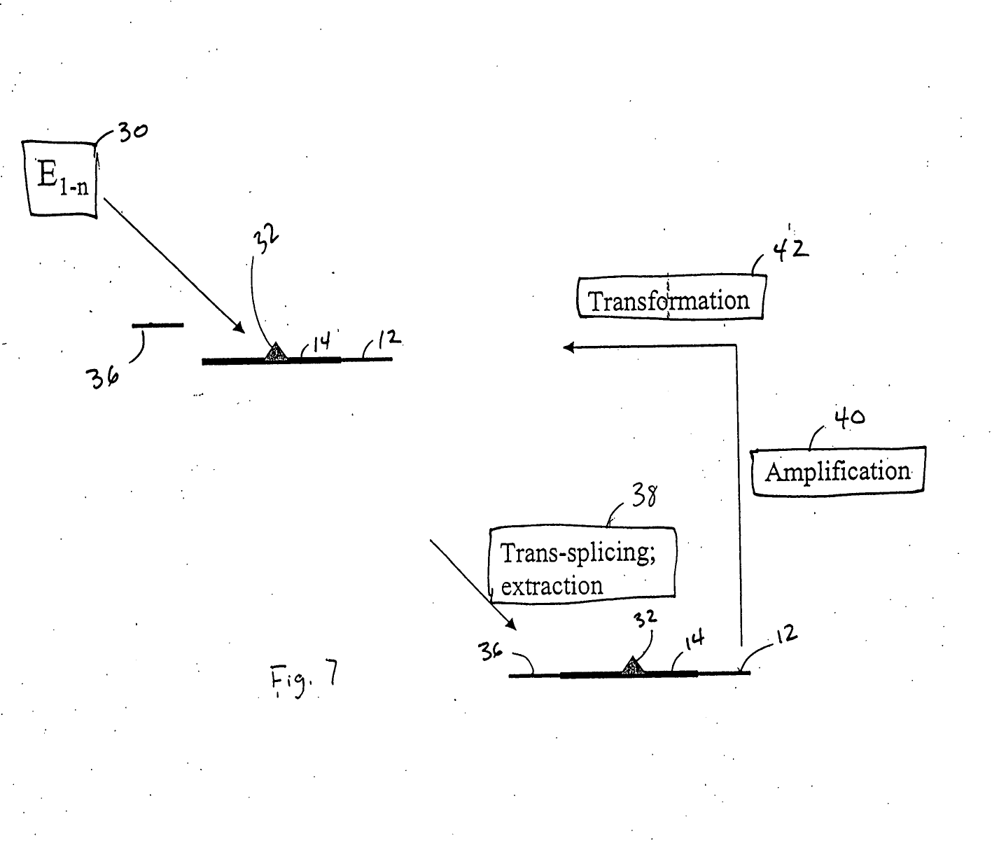

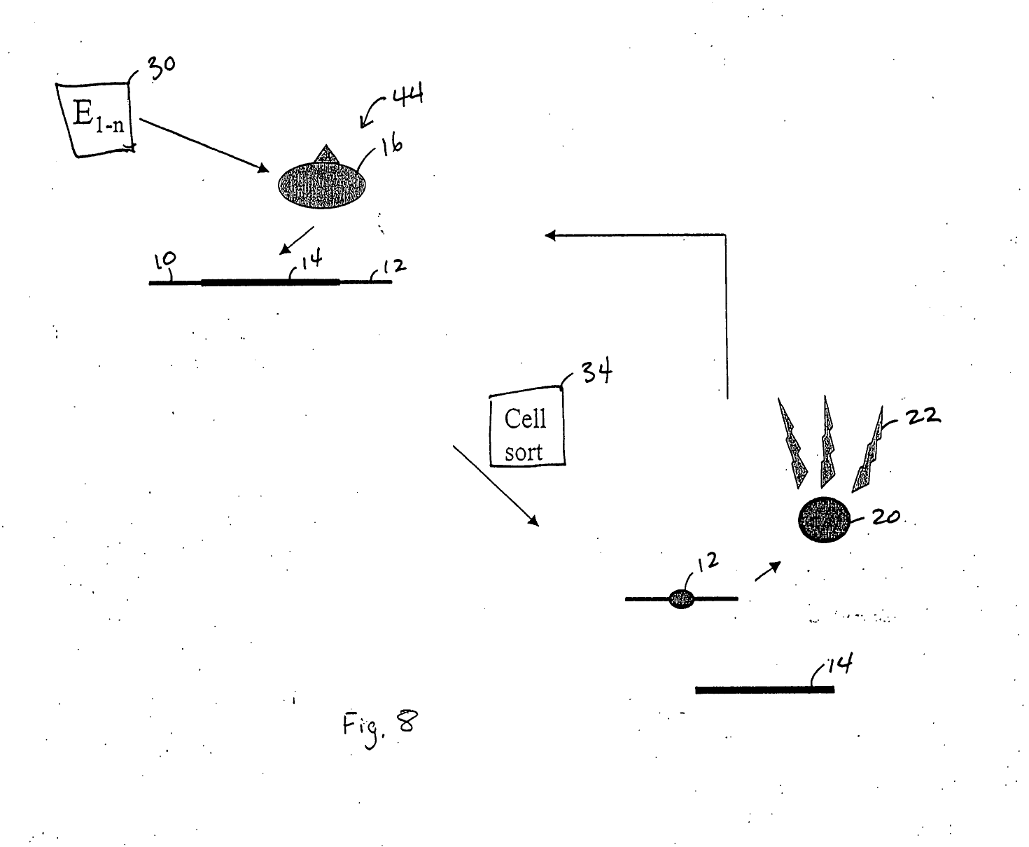

FIG. 7 depicts an alternative embodiment for screening libraries of exogenous activators. In the embodiment of the present invention of FIG. 7, Group I introns with length polymorphisms are induced into the construct by trans-splicing with an independent oligonucleotide. Libraries of candidate exogenous activators 30 can be generated from a randomized aptazyme pool indicated by random loop E₁₋ₙ. As in the embodiment of FIG. 5, a reporter gene 12 is expressed in cells where the exogenous activator 30 activates the aptazyme to release the intron, which may contain a random loop 32, from the gene. In this embodiment, the reaction occurs within the intron 14 and an independent oligonucleotide 36 by a trans-splicing reaction and extraction step 38. Extracted trans-spliced intron reporter gene constructs are then amplified by, e.g., polymerase chain reaction in step 40, followed by transformation of cells with the transpliced construct at step 42. Transformation of the transpliced construct may be performed by those of skill in the art with either a negative or positive selection scheme for identification of the trans-spliced gene.

FIG. 8 shows yet another alternative embodiment for screening libraries of exogenous activators 30 with the present invention. In the embodiment of FIG. 8, a pool of randomized loops 32 interact with an effector 16 (or a second exogenous effector) to form a complex 44. The complex 44 is exposed to the gene 10 containing both the Group I intron 14 and a reporter gene 12. Cell sorting 34 reveals the cells that express the reporter gene 20 to indicate successful activation of the aptazyme by the effector.

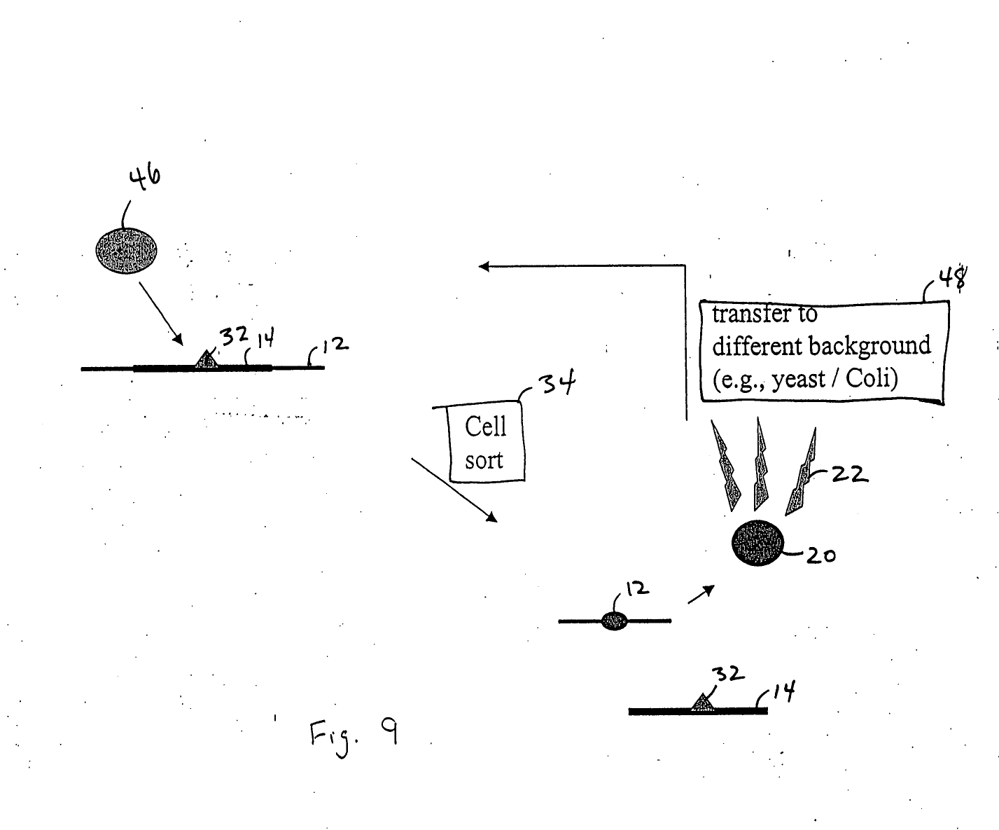

FIG. 9 is a diagram of an embodiment of the present invention for screening for endogenous suppressors. In this embodiment, an endogenous effector 46, in this illustration shown as a protein suppressor from endogenous or transformed origin, activates self-splicing of the Group I intron 14 (depicted with a random loop 32). Cell sorting 34 is used to reveal the expression of the reporter gene. To protect against spontaneous auto-excision of the intron, the gene may be transferred into a different background system (at step 48) such as yeast or E. coli, for example.

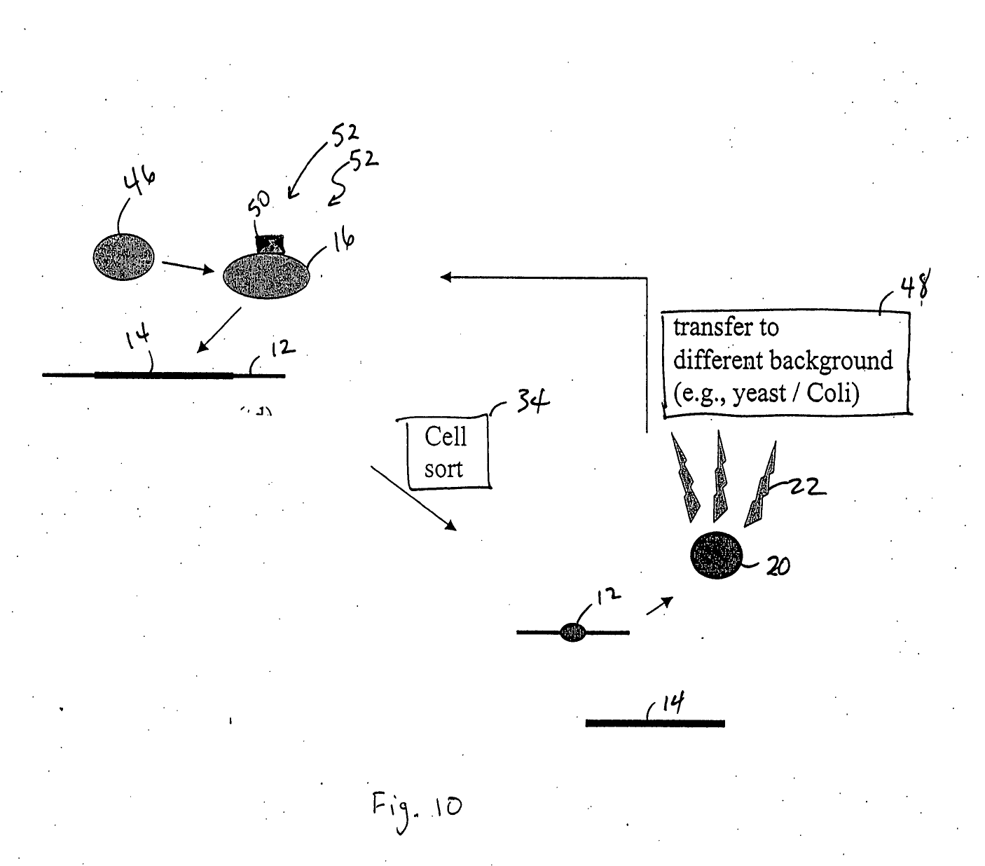

FIG. 10 depicts an alternative to the embodiment of FIG. 9 to screen for endogenous activators of the present invention. In FIG. 10, the activator that is being screened for may include, inter alia, a phosphorylated protein, a product of ubiquitination, or a protein-protein complex. For example, a protein suppressor 46, may phosphorylate 50 an effector molecule 16 (e.g., Cytl8). The phosphorylated effector molecule 52 activates intron 14 self-splicing with concomitant expression of the reporter gene 12, e.g., green fluorescent protein (GFP).

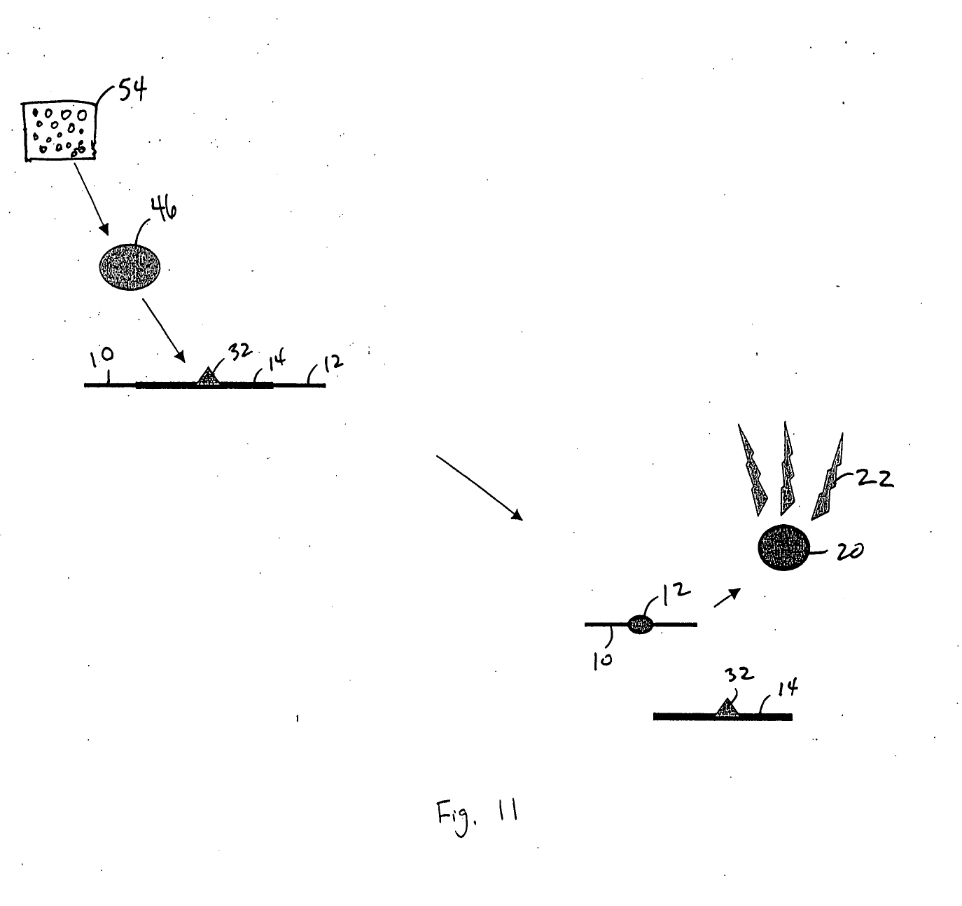

FIG. 11 shows yet another embodiment of the present invention to screen for compounds that perturb cellular metabolism. In this embodiment, a communication module pool 54, undergoes selection for a phenotype responsive to a protein suppressor 46 effector molecule. The protein effector 46 may be a phosphoprotein, an induced protein, or a protein complex, for example. The source of the effector may be endogenous, exogenous or may even be the product of a transformation construct used to transform a cell. Activation by the effector results in expression of the reporter gene 12, but may inactivate, suppress, or “knocks-out” the gene 10 of interest. The functioning of the gene of interest may thereby be perturbed, providing information about the function and/or regulation of the gene or gene product.

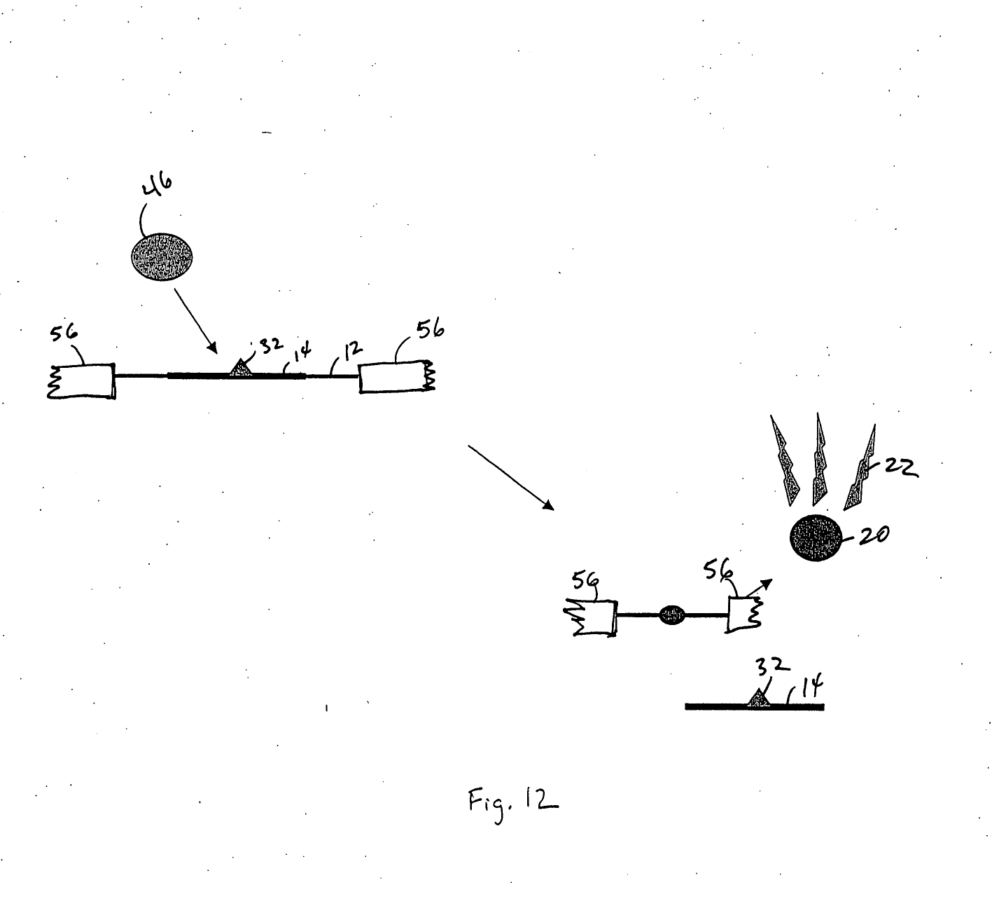

FIG. 12 shows a further embodiment of the present invention that provides a non-invasive readout of metabolic states. An aptazyme construct of the present invention may be introduced into a gene of interest 56 that forms part of the host (whether chromosomal or extrachromosomal). A protein suppressor 46 from either an endogenous or exogenous source is used to screen for the product of a cell transformation that may activate self-splicing of the aptazyme, leading to expression of the reporter gene 10. Whether or not the gene of interest is expressed upon release of the aptazyme intron from the gene provides information about the metabolic state of the gene of interest. The embodiment of the present invention of FIG. 12 thus, provides a non-invasive means to determine the metabolic state of an organism with regard to a gene of interest.

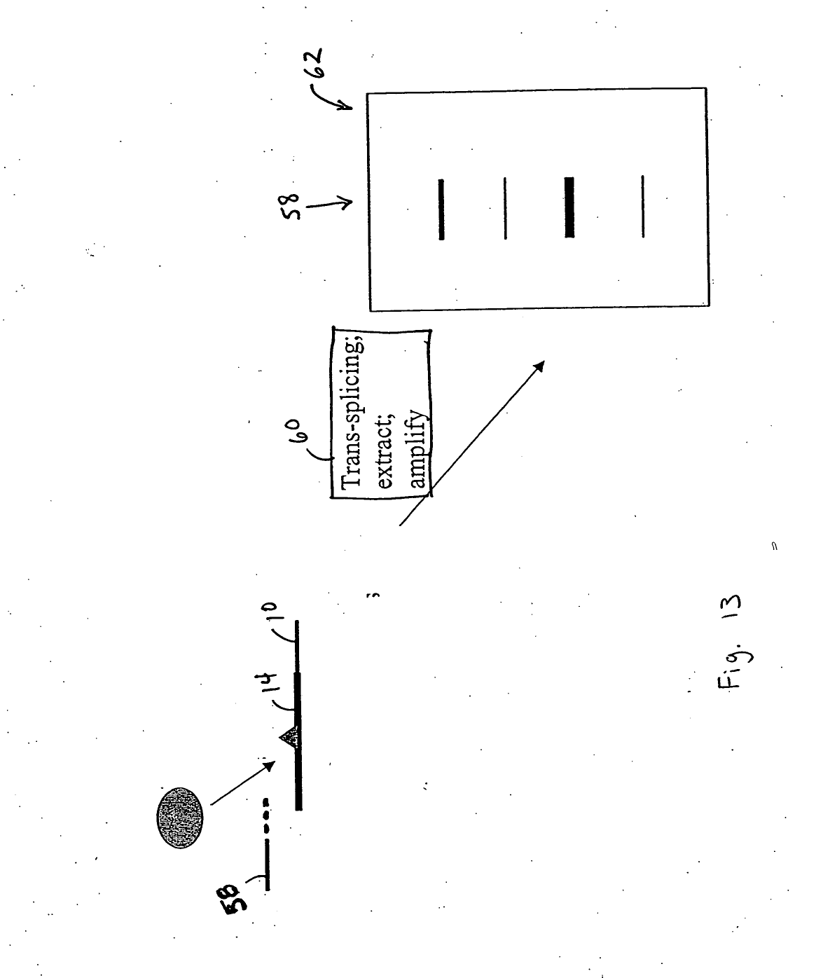

FIG. 13 depicts a further embodiment of the present invention wherein endogenous suppressors provide a non-invasive readout of multiple metabolic states. Multiple protein suppressors 46 (endogenous or transformed) are exposed to a pool of Group I introns 14 of the present invention. The pool includes introns with length polymorphisms that are depicted in FIG. 13 by a discontinuity or break in the line representing the Group I intron 14 residing in a gene of interest 10. Activation of the aptazyme leads to trans-splicing among the various polymorphisms 58. The products of trans-splicing may be extracted and amplified in step 60. Separation of the trans-splicing products 58 by gel electrophoresis 62 provides a read out of the protein function or the metabolic pathway. The readout may even be digitized for analysis.

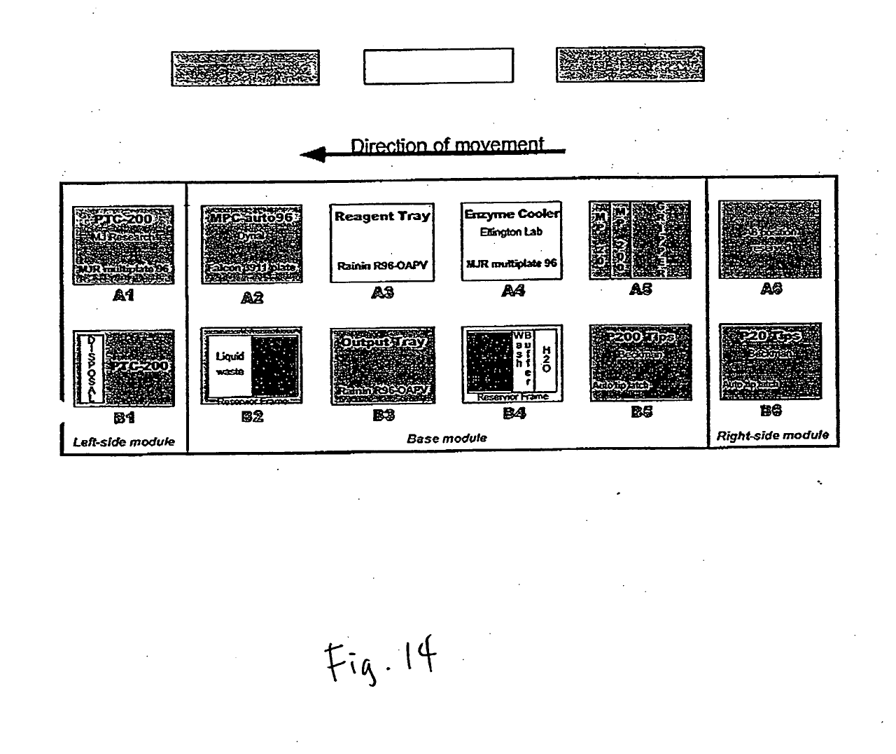

FIG. 14 depicts schematically an exemplary worksurface for yet another embodiment of the present invention: automated selection. See, J. C. Cox, et al., Automated RNA Selection Biotechnol. Prog., 14, 845 850, 1998.

Base protocol. Automated selection involves several, sequential automated steps. Several modules are placed on the robotic worksurface, including a magnetic bead separator, and enzyme cooler, and a thermal cycler. After manually preparing reagents and preloading those reagents (including random pool RNA, buffers, enzymes, streptavidin magnetic beads, and biotinylated target) and tips onto the robot, a program is run. The selection process, automated by the robot, goes as follows: RNA pool is incubated in the presence, of biotinylated target conjugated to streptavidin magnetic beads. After incubation, the magnets on the magnetic bead separator are raised, and the beads (now bound by pool RNA—the selected aptamers) are pulled out of solution. Thus, the beads can be washed, leaving only RNA bound to targets attached to beads.

These RNA molecules are reverse transcribed, reamplified via PCR, and the PCR DNA is in vitro transcribed into RNA to be used in iterative rounds of selection.

The Bioworks method for in vitro selection. This scripted programming method contains all movements necessary in order to facilitate automated selection. This includes all physical movements to be coordinated, and also communication statements. For instance, five rounds of automated selection against a single target requires over 5,000 separate movements in x, y, z, t coordinate space. Additionally, the method also holds all relevant measurements, offsets, and integrated equipment data necessary to prevent physical collisions and permit concerted communication between devices.

“Beads on filter” selections. While the vast majority of manual selections have been performed on nitrocellulose-based filters, a small few have also been performed on solid surfaces, such as beads. We have developed a novel selection scheme whereby selection is performed on magnetic beads that are placed on nitrocellulose filters, and washed as the bead is the selection target itself. This method allows for much greater specificity of selection, thereby promoting ‘winning’ molecules to amplify in greater number, and thus reduce the overall amount of rounds necessary to complete the selection procedure. Manual selection does not involve a combination of surfaces to enhance selection.

Cross-contamination avoidance. The introduction of contaminating species of nucleic acid strands in a manual selection can be commonplace. This is especially true if selection is done against multiple targets in parallel, and also when a researcher reuses the same pool for different selection tools. Contaminating species have been shown in the past to interfere with a manual selection such that it could not be completed. Automated in vitro selection takes steps to minimizing and/or eliminate the possibility of cross-contamination between pools and targets. Movement of the mechanical pod along the worksurface is unidirectional when carrying potentially contaminating material. This movement away from ‘clean’ things and only towards items that have already been exposed to replicons greatly diminishes the possibility of cross-contaminating reactions. The only circumstance in which the pod reverses its direction is to acquire a new, clean pipette tip. Additionally, we have also sealed our reagent trays with aluminum foil for a physical barrier between the environment and unexposed reagents. See FIG. 14, a layout of the robotic worksurface which reduces cross-contamination.

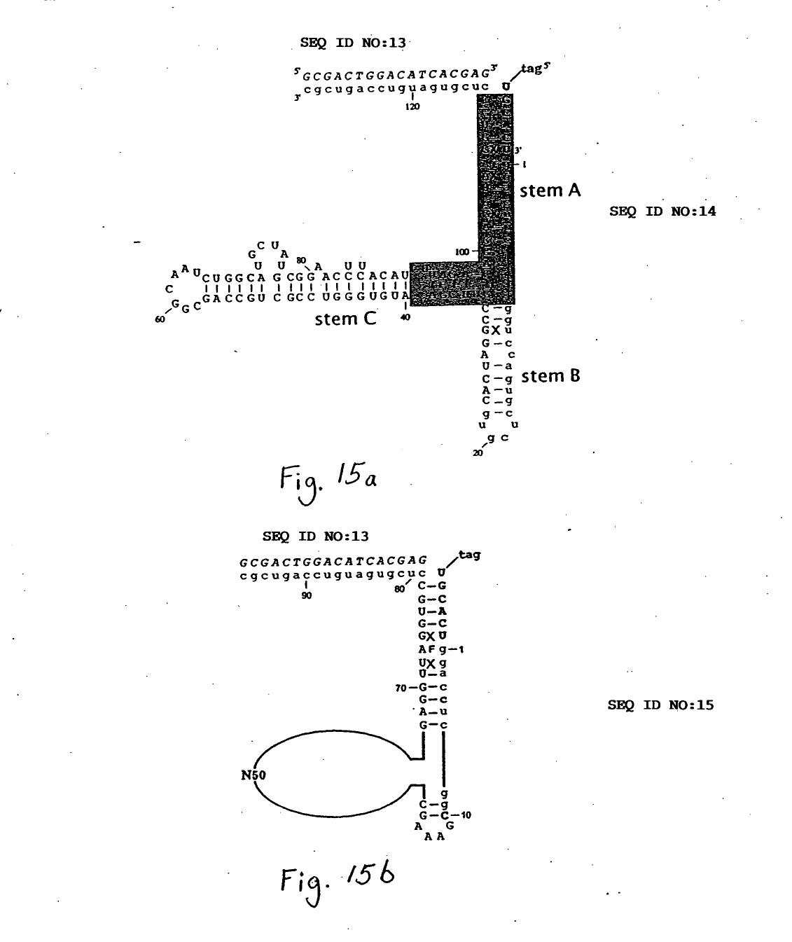

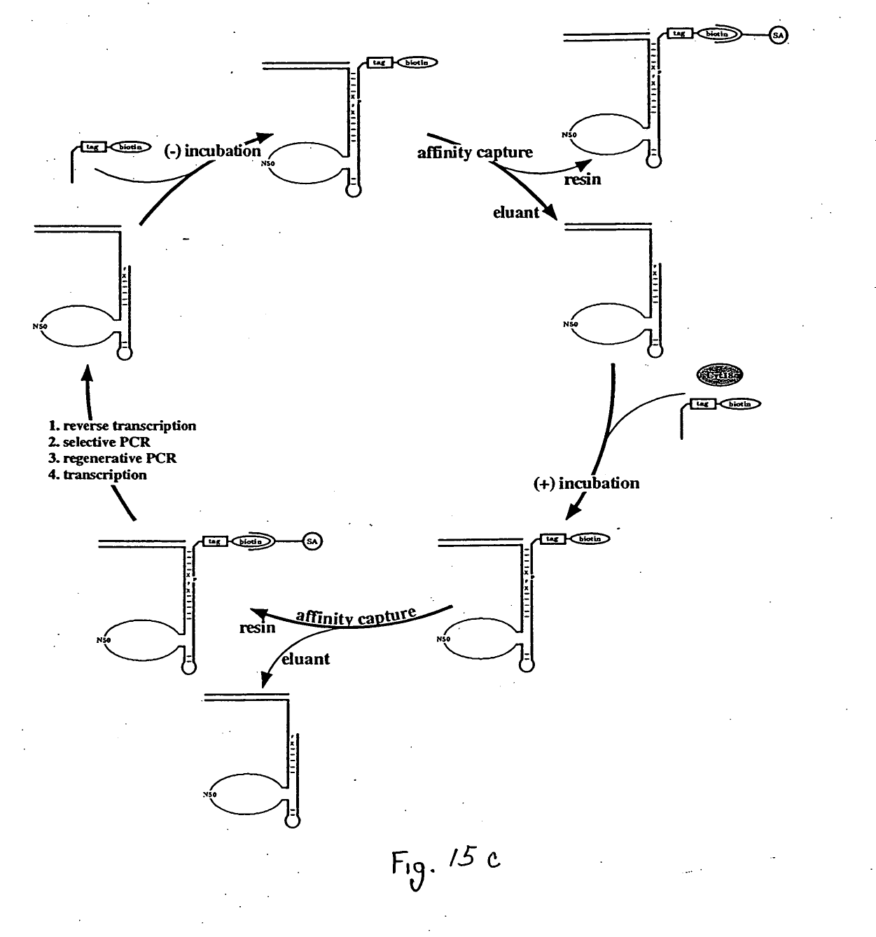

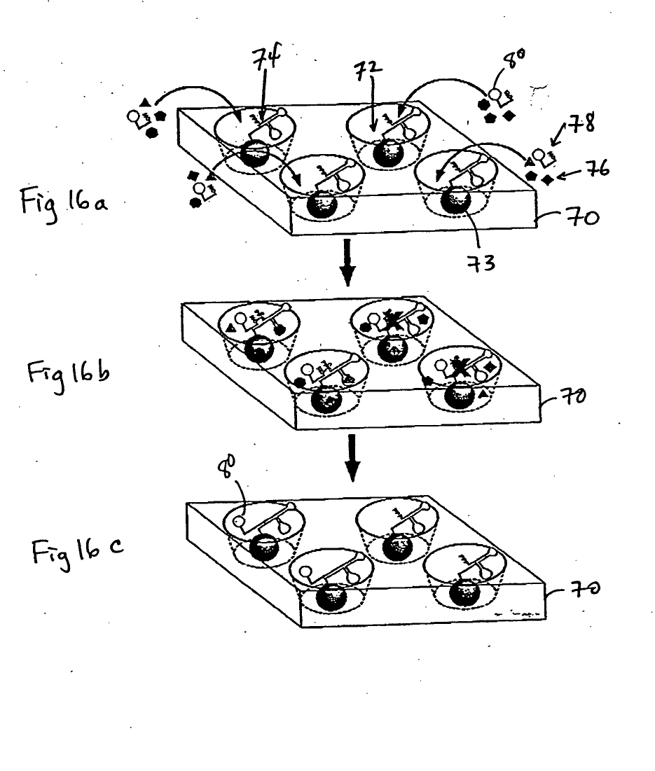

FIG. 15a depicts the LI ligase used for pool design in, e.g., the Cytl8 aptazyme selection, as an example of a protein-activated aptazyme. Stems A, B, and C are indicated. The shaded region contains the catalytic core and ligation junction. Primer binding sites are shown in lower case, an oligonucleotide effector required for activity is shown in italics, and the ligation substrate is bolded. The ‘tag’ on the ligation substrate can be varied, but was biotin in the exemplary selection described herein. The LI pool contains 50 random sequence positions and overlaps with a portion of the ribozyme core. In FIG. 15b, Stem B was reduced in size and terminated with a stable GNRA tetraloop, but stem A was unchanged.

FIG. 15c schematically shows the following selection scheme: the RNA pool was incubated with a biotinylated tag and reactive variants were removed from the population. The remaining species were again incubated with a biotinylated tag in the presence of the target (Cytl8). Reactive variants were removed from the population and preferentially amplified by reverse transcription, PCR, and in vitro transcription.

FIGS. 16a through 16c schematically depict one method to anchor aptazymes. A substrate 70, e.g, glass, silicon, gallium arsenide, silicon on insulator (SOI) structures, epitaxial formations, germanium, germanium silicon, polysilicon, amorphous silicon, and/or like substrate, semi-conductive or conductive is depicted. The substrate 70 may already have been processed to provide electrical means of detection on the surface of the wells 72. One example of such a detector is a charge coupled capacitor.

In FIG. 16a, different ribozyme ligases 74 are shown immobilized on beads 73 in wells 72, and mixtures of analytes 76 and tagged substrates 78 may be added to each well. Next, in FIG. 16b, cognate effectors are present (same analyte and allosteric site) in the well 72 and the aptazymes 74 will covalently attach the reporter tags 80 (e.g., fluorescent tags) to themselves. Where aptazymes 74 have not been activated by effectors, the tagged substrates 78 are washed is out of the well. In FIG. 16c, after reaction and washing, the presence and amounts of co-immobilized reporter tags 80 are indicative of amounts of ligands that were present during the reaction. See K. A. Marshall, et al., Training Ribozymes to Switch, Nature Struct. Biol. 6 (11) 992-994, 1999.

In the embodiment of FIG. 16, the reporter tag 80 may be an enzyme, a magnetic particle, or any number of detectable particles. Additionally, the ribozymes could be immobilized on beads 83, but they could also be directly attached to a solid support or substrate 70 via covalent bonds.

One advantage of this embodiment is that covalent immobilization of reporters allows stringent wash steps to be employed. This can be distinguished from non-covalent immobilization assays such as ELISA™ assays where stringent washing may destroy the signal. An additional advantage is that ribozyme ligases have the unique property of being able to transduce effectors into templates that can be amplified, affording an additional boost the in signal prior to detection.

Although nucleic acids are generally less robust than antibodies, modified nucleotides may be introduced into the aptazymes that substantially stabilize them from degradation in environments such as sera or urine. Antibodies generally have higher affinities for analytes than do aptamers. However, the analytical methods of the present invention do not rely on binding per se, but only on transient interactions. The present invention requires mere recognition rather that actual binding, thus providing a potential advantage of aptazymes over antibodies. That is, even low affinities are sufficient for activation and subsequent detection, especially if individual immobilized aptazymes are examined (i.e., by ligand-dependent immobilization of a quantum dot).

All publications mentioned in the above specification are hereby incorporated by reference. Modifications and variations of the described compositions and methods of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described compositions and modes of carrying out the invention which are obvious to those skilled in molecular biology or related arts are intended to be within the scope of the following claims.

1. A method for detecting an aptazyme reaction, the method comprising the steps of:
providing a solid support having a heterogeneous mixture of aptazyme constructs covalently immobilized thereon;
providing at least one analyte;
providing a nucleic acid substrate tagged to be detectable;
exposing the nucleic acid substrate and at least one analyte to the immobilized aptazymes, whereby activation of the aptazyme reaction by the analyte produces a signal when the nucleic acid substrate is bound to the immobilized aptazymes;
washing unbound substrate off the solid support; and
detecting the signal from the bound nucleic acid substrate.

2. The method of claim 1, wherein the method is automated.

3. The method of claim 1, wherein the signal is amplified for detection.

4. The method of claim 1, wherein the nucleic acid substrate tagged to be detectable is fluorescently tagged, tagged with a magnetic particle, or tagged with an enzyme.

5. The method of claim 1, wherein the solid support is a bead or a well in a multiwell plate.

6. The method of claim 5, wherein the solid support is a bead in a well of a multiwell plate.

7. The method of claim 6, wherein each well contains a bead with an aptazyme construct immobilized thereto which is different from the aptazyme constructs immobilized on the beads located in the other wells of the multiwell plate.

8. The method of claim 1, wherein the analyte is a metabolite or a protein.

9. A method for detecting an aptazyme reaction, the method comprising the steps of:
providing a solid support having an aptazyme construct covalently immobilized thereon;
providing at least one analyte;
providing a nucleic acid substrate tagged to be detectable;
exposing the nucleic acid substrate and at least one analyte to the immobilized aptazyme, whereby activation of the aptazyme reaction by the analyte produces a signal when the nucleic acid substrate is bound to the immobilized aptazyme;
washing unbound nucleic acid substrate off the solid support; and
detecting the signal from the bound nucleic acid substrate.

10. The method of claim 9, wherein the method is automated.

11. The method of claim 9, wherein the signal is amplified for detection.

12. The method of claim 9, wherein the nucleic acid substrate tagged to be detectable is fluorescently tagged, tagged with a magnetic particle, or tagged with an enzyme.

13. The method of claim 9, wherein the solid support is a bead or a well in a multiwell plate.

14. The method of claim 13, wherein the solid support is a bead in a well of a multiwell plate.

15. The method of claim 9, wherein the analyte is a metabolite or a protein.

16. A method for detecting an analyte in a sample suspected of containing said analyte by detecting the binding of an aptazyme to a substrate, the method comprising the steps of:
providing an array having one or more aptazyme constructs disposed thereon at discrete locations by immobilization of said aptazyme constructs on a solid support;
contacting said aptazyme constructs with a substrate tagged with a detectable label, wherein said aptazyme constructs bind to said tagged substrate in the presence of said analyte, but do not bind to said tagged substrate in the absence of said analyte;
contacting said aptazyme constructs and substrate with in a sample suspected of containing said analyte under conditions which allow for substrate binding;
washing away unbound substrate;
detecting the bound substrate, thereby determining the presence of analyte in said sample.
